(19) United States
(12) Patent Application Publication
(10) Pub. No.: US 2006/0045861 A1
(43) Pub. Date: Mar. 2, 2006

(54) Reduced odor in low molecular weight cationic polygalactomannan

(76) Inventors: Thomas P. Bejger, Odessa, DE (US); Paquita Erazo-Majewicz, Newark, DE (US); Daniel L. Hopkins, Hockessin, DE (US); John N. Kostas, Wilmington, DE (US); Pong-Kuen P. Kuo, Hockessin, DE (US); Jashawant J. Modi, Hockessin, DE (US); Zu-Feng Xu, Newark, DE (US)

Correspondence Address: HERCULES INCORPORATED;HERCULES PLAZA, 1313 NORTH MARKET STREET, WILMINGTON, DE, 19894-0001, US

(21) Appl. No.: 11/202,469

(22) Filed: Aug. 12, 2005

Related U.S. Application Data
(60) Provisional application No. 60/605,556, filed on Aug. 31, 2004.

(51) Int. Cl. A61K 8/73

(52) U.S. Cl. 424/70.13

(57) ABSTRACT

A reduced odor composition is composed of at least one cationic polygalactomannan or a derivative of cationic polygalactomannan having a weight average molecular weight (Mw) having a lower limit of 5,000 and an upper limit of 200,000, a light transmittance in a 10% aqueous solution of greater than 80% at a light wavelength of 600 nm, a protein content of less than 1.0% by weight of polysaccharide, and a trimethylamine content of less than 25 ppm in a 10% aqueous solution of the polymer. This composition is prepared by treating the polymer with reagents that reduce the molecular weight of the polymer, removing the water-insoluble solid material, and removing odorous components, including trimethylamine (TMA) and other amines and low molecular weight components from the aqueous phase to produce a polymer that when used in a functional system such as household care, personal care or pet care products has reduced or no odor at acidic, neutral, or alkaline pH values.

This application claims the benefit of U.S. Provisional Application No. 60/605,556, filed Aug. 31, 2004.

### FIELD OF INVENTION

The present invention is related to a polygalactomannan composition and more particularly, to a guar gum composition which when dispersed in water is capable of forming a relative transparent solution, uses of this polygalactomannan composition in personal care, household, industrial and institutional compositions that have no discernible amine odor at acidic, neutral, or alkaline pH values, and processes for producing this polygalactomannan composition.

### BACKGROUND OF THE INVENTION

Low and high molecular weight cationic galactomannan polymers are used as conditioners in personal cleansing products such as shampoos and body washes, which are typically formulated at acidic or neutral pH values. As a result of processing, an amine odor is apparent in samples of some cationic galactomannan polymers such as cationic guars. At acidic and neutral pH values there is no apparent objectionable “fishy” odor, characteristic of amines, such as trimethylamine (TMA). This is expected, since at acidic or neutral pH values, most amines are in the aqueous phase, in the nonvolatile salt form. Consequently, there has been no critical need identified for a low odor version of low or high molecular weight cationic galactomannan or cationic guar polymers. However, on incorporation of some cationic galactomannan polymers such as cationic guars in personal cleansing products and in household products, such as detergents and fabric conditioners, formulated at alkaline pH values, an unacceptable odor develops that is characteristic of an amine.

Cationic polysaccharides and other polymers have been used widely in personal care, household, industrial, and institutional products to perform a function in the final product, ranging from the use of the polymer as gellants, binders, thickeners, stabilizers, emulsifiers, spreading and deposition aids and carriers for enhancing the rheology, efficacy, deposition, aesthetic and delivery of chemically and physiologically active ingredients in personal care, household, institutional and industrial compositions. Depending on the application, the substrate to which the product is applied can be skin, hair, or textile substrates.

Cationic polysaccharides are used in hair care products to provide conditioning to the hair. In skin care products, these same polymers can provide conditioning effects to the skin. When incorporated into detergent and fabric softening formulations, these same polymers can provide conditioning, softening, anti-abrasion and antistatic characteristics to fabrics.

Wet and dry combability measurements are typical test methods used to measure conditioning performance in shampoo and conditioner applications. Commercial cationic conditioning polymers in the marketplace have been reported to reduce the wet combing force experienced on combing wet hair by 30%-80% relative to the shampoo containing no polymer. The performance of different cationic polymers in these applications varies, however, in achieving a good balance of wet and dry combing force reduction, with good optical clarity in a formulation. EP1501873 A1 addresses this need for a cationic galactomannan polymer with good optical clarity in personal care, household, and fabric cleansing formulations.

A need still exists in the marketplace, for a cationic galactomannan conditioning polymer that has broad surfactant compatibility, which can deliver clear personal care, household formulations, institutional, and industrial formulations with good conditioning performance with no discernible amine odor at alkaline pH values.

A need also exists in the marketplace for a process that removes amine and other undesired low molecular weight components from polygalactomannan compositions.

### SUMMARY OF THE INVENTION

The present invention is directed to a composition of at least one cationic polygalactomannan or cationic derivatized galactomannan polymer having a weight average molecular weight (Mw) having a lower limit of 5,000 and an upper limit of 200,000 and having a light transmittance in a 10% aqueous solution of greater than 80% at a wavelength of 600 nm, a protein content of less than 1.0% by weight of polymer, and a level of trimethylamine of less than 25 ppm in a 10% aqueous solution of the polymer.

This composition can optionally have an aldehyde functionality content of at least 0.01 meq/gram and/or a boron content of less than 50 ppm per gram of polygalactomannan.

This invention is further directed to a process for preparing the composition mentioned above including the steps of a) reacting at least one cationic galactomannan polymer or derivatized cationic galactomannan polymer with at least one reagent that reduces the weight average molecular weight (Mw) of the galactomannan polymer in the reaction mass to less than 200,000 wherein the reaction mass also includes water-soluble color bodies and water-insoluble materials, b) removing the water-insoluble solid materials, and c) applying a process where odorous components including trimethylamine (TMA) and low molecular weight components are removed or reduced to produce the cationic polygalactomannan composition of the invention.

This invention is further directed to a composition of a functional system of personal care products, household care products, and pet care products containing the above mentioned cationic polygalactomannan composition and optionally at least one active personal care, household care, or pet care ingredient, respectively.

### DETAILED DESCRIPTION OF THE INVENTION

It has been found unexpectedly that a low odor cationic galactomannan polymer composition can be produced by application of various methods that reduce the amine content, such as trimethylamine content, in aqueous solutions of cationic galactomannan polymers. It has been found, also, that there is a significant reduction in odor in personal care and household products incorporating the low odor cationic galactomannan polymer compositions of the invention at alkaline pH values.

In addition, the polymers of this invention may possess other attributes including their ability to deliver clear formulations across a range of surfactant systems and across a range of polymer concentrations, in personal care and household products. The polymers of this invention can also deliver conditioning effects with high clarity in personal care products and in other surfactant-based products, such as household products.

In accordance with the invention, the polymer of the invention imparts no malodor or discernible amine odor to personal care, household, or other products when formulated at acidic, neutral, or alkaline pH values when the polymer of the invention is incorporated into the formulation at a level of less than 10 wt %, preferably at a level of less than 5 wt %, and more preferably at a level of less than 1 wt %.

In accordance with the invention, the polymers that can be used in the invention include cationic galactomannan polymers or cationic derivatized galactomannan polymers having a weight average molecular weight (Mw) having a lower limit of 5,000 preferably 20,000, more preferably 35,000, and most preferably 50,000. The upper limit of the Mw of these polymers is less than 200,000, preferably 100,000, and more preferably 70,000. Examples of the polygalactomannans of this invention are guar, locust bean, honey locus, and flame tree with guar gum being the preferred source of the polygalactomannan. The preferred polygalactomannan starting material used in this invention is guar flour, guar powder, guar flakes, guar gum, or guar splits which has been derivatized with a cationic substituent.

The preferred polymers of this invention are cationic polygalactomannan polymers. The amount of cationic functionality on the polygalactomannan can be expressed in terms of moles of substituent. The term “degree of substitution” as used in this invention is equivalent to the molar substitution, the average number of moles of functional groups per anhydro sugar unit in the polygalactomannan gum. The cationic functionality can be present on these polymers at a DS level as low as 0.01, preferably about 0.1, and more preferably 0.2. The DS upper limit is normally about 3.0, preferably about 2.0, and more preferably 1.0. In addition to molar substitution, the cationic charge on the polymers of this invention can be quantified as a charge density. The molar substitution can be converted to a charge density through a variety of methods. The preferred method for calculating charge density of cationic polymers uses a method that specifically quantifies the equivalents of quaternary ammonium groups on the polymer. Starting material having a cationic molar substitution level of 0.18 has been determined to have a charge density of 0.95 mequivalents per gram (meq/g) according to the following equation:
Cationic charge density of DS 0.18 cationic guar=(1000×0.18)/(162.14+(151.64×0.18))=0.95 meq/g.

Charge density can be measured by any method that quantifies the net positive or negative charge present on a polymer. The charge density can be determined by measurement of the moles of quaternary ammonium groups bound to the polymer backbone using standard NMR techniques of integration. This method was used for determining the charge density for polymers of this invention.

The cationic functionality of the polygalactomannan or derivatized polygalactomannan can be added to them by several methods. For example, the starting material can be reacted for a sufficient time and at a sufficient temperature with tertiary amino compound or quaternary ammonium compound containing groups capable of reacting with the reactive hydrogen ions present on the polygalactomannan or derivatized polygalactomannan in order to add the cationic functionality to the starting material. The sufficient time depends on the ingredients in the reaction mass and the temperature under which the reaction is taking place.

The canonizing agent of the present invention is defined as a compound which, by substitution reaction with the hydroxy groups of the polygalactomannan can make the product electrically positive, and there is no limitation to its types. Tertiary amino compounds or various quaternary ammonium compounds containing groups capable of reacting with reactive hydrogen present on the polysaccharide, can be used, such as 2-dialkylaminoethyl chloride and quaternary ammonium compounds such as 3-chloro-2-hydroxypropyltrimethylammonium chloride, and 2,3-epoxy-propyltrimethylammonium chloride. Preferred examples include glycidyltrialkylammonium salts and 3-halo-2-hydroxypropyltrialkylammonium salts such as glycidyltrimethylammonium chloride, glycidyltriethylammonium chloride, gylcidyltripropylammonium chloride, glycidylethyldimethylammonium chloride, glycidyldiethylmethylammonium chloride, and their corresponding bromides and iodides; 3-chloro-2-hydroxypropyltrimethylammonium chloride, 3-chloro-2-hydroxypropyltriethylammonium chloride, 3-chloro-2-hydroxypropyltripropylammonium chloride, 3-chloro-2-hydroxypropylethyldimethylammonium chloride, and their corresponding bromides and iodides; and quaternary ammonium compounds such as halides of imidazoline ring containing compounds.

Other derivatization of the cationic polygalactomannan with nonionic substituents, i.e., hydroxyalkyl wherein the alkyl represents a straight or branched hydrocarbon moiety having 1 to 6 carbon atoms (e.g., hydroxyethyl, hydroxypropyl, hydroxybutyl) or anionic substituents, such as carboxymethyl groups are optional. These optional substituents are linked to the polygalactomannan molecule by the reaction of the polygalactomannan molecule with reagents such as (1) alkylene oxides (e.g., ethylene oxide, propylene oxide, butylene oxide) to obtain hydroxyethyl groups, hydroxypropyl groups, or hydroxybutyl groups, or with (2) chloromethyl acetic acid to obtain a carboxymethyl group on the polygalactomannan. This reaction can take place when the polygalactomannan is in the “split”, “flour” or any other physical form. The process for preparing derivatized polygalactomannan is well known in the art.

In accordance with this invention, the cationic polygalactomannan or cationic derivatized polygalactomannan composition not only has a reduced viscosity and low weight-average molecular weight (Mw) but also has a percent light transmittance in a 10% aqueous solution of greater than 80% at a wave length of 600 nm, preferably greater than 90%, and more preferably greater than 95%.

In accordance with this invention, the cationic polygalactomannan or cationic derivatized polygalactomannan composition has a trimethylamine content in a 10% aqueous solution of less than 25 ppm, preferably less than 7 ppm, and most preferably less than 5 ppm when measured by any method known to those skilled in the art. Examples of methods used to measure trimethylamine include gas chromatography (GC), mass spectrometry, solid phase extraction methods using fiber adsorbents, and combinations thereof.

In accordance with this invention, the low molecular weight polygalactomannan has low protein contents. While conventional polygalactomannan gum may have about 3% protein content, as measured by quantification of percent nitrogen or by use of colorimetric techniques (M. M. Bradford, Anal. Biochem., 1976, 72, 248-254), the polygalactomannan compositions of this invention have a protein content of less than 1% as measured by the Bradford method, and preferably less than 0.5%.

Borate salts are commonly used to cross-link guar prior to derivatization. These borate salts generally remain strongly bound to the cationic polygalactomannan product even after water washing. The process steps used to reduce the trimethylamine content of the cationic polygalactomannan polymers of the invention also readily removes borate salts from the polymer, producing a higher purity cationic polygalactomannan. In the present invention, the boron content of the polygalactomannan is less than 50 ppm, preferably less than 30 ppm, and more preferably less than 10 ppm per gram of polygalactomannan.

In accordance with the present invention, the molecular weight of polygalactomannans can be reduced as set forth in step a above, by several different methods, such as (1) by biochemical methods wherein polysaccharide hydrolytic enzymes, bacteria, or fungi are used directly, (2) chemical method using (a) acid (b) alkali, or (c) through the use of oxidative agents, i.e., hydrogen peroxide, (3) physical methods using high speed agitation and shearing machines, (4) thermal methods, or (5) depending on necessity, a suitable purification method can be used to make the molecular weight fall within a certain range. Examples of the purification methods that can be used are filtration using a filter-aid, ultrafiltration, reverse osmosis membrane, selective density centrifugation, and chromatography.

In accordance with this invention, an oxidative reagent either alone or in combination with other reagents, including biochemical reagents, is used to reduce molecular weight or introduce oxidized functional groups. In order to achieve optimum results, it is necessary to include the oxidative reagent in the process either completely or alternately with other reagents.

Oxidative agents include any reagent that incorporates oxygen atoms into the polymer structure. Some oxidizing reagents can also act to reduce the molecular weight of the polymer. Examples of these dual function oxidizing agents are peroxides, peracids, persulfates, permanganates, perchlorates, hypochlorite, and oxygen. Examples of biochemical oxidative agents that do not reduce molecular weight but introduce aldehyde functionality are oxidases. Specific examples of oxidases useful in this invention are galactose oxidase, and other biochemical oxidizing agents known to those skilled in the art.

A generalized preferred process for producing the cationic polygalactomannan or derivative of the cationic polygalactomannan composition is as follows:
(a) reacting a small portion of the cationic polygalactomannan or derivative with an oxidizing reagent or a combination of a hydrolytic reagent and an oxidizing reagent in the presence of water for a sufficient time to reduce the viscosity and molecular weight of the polymer;
(b) adding additional quantities of the polymer and oxidizing reagent making multiple additions of the polymer and oxidizing agent depending on the desired results and the reaction parameters;
(c) terminating the reaction and recovering a fluid aqueous dispersion of the composition that contains water soluble color bodies, and water insoluble material, and water at a concentration of about 50 to 95% by weight of the total composition;
(d) removing water insoluble material from the aqueous dispersion to produce a clarified, aqueous solution of the composition of this invention. Conventional means are used for removing the water insoluble materials, such as centrifugation and filtration methods; and
(e) removing of odorous components, including trimethylamine (TMA) and other amines and low molecular weight components in the aqueous phase to produce the cationic polygalactomannan composition of the invention.

Optionally, this process can include an additional step to remove the water soluble color bodies to produce a colorless, clarified aqueous solution of the composition of this invention. Examples of reagents and materials that can be used to remove the color bodies include sodium bisulfite, sodium metabisulfite, sodium hypochlorite, sodium chlorite, activated carbon, and molecular sieves.

When the combination of the hydrolytic reagent and an oxidizing reagent is used in this invention, the oxidizing reagent will be used in step (b) and the hydrolytic reagent will be used in step (a). This alternating of reagents can be used throughout the process. In another embodiment, all of the hydrolytic reagent and polymer are added batchwise to the reaction vessel and the reaction is allowed to continue to the desired viscosity. If the hydrolytic reagent is an enzyme, it is then deactivated by heat at the end of the reaction. Thereafter, the reaction mass is clarified to a clear solution by conventional processes. An oxidizing reagent is added to the clarified solution and reacted to the desired viscosity and molecular weight for the final product.

Alternatively, the reaction can be performed in a batch process with one addition of reagent (either dual function or combination of hydrolytic reagent and oxidizing reagent) at the beginning of the reaction, with a content of polygalactomannan solids that allows for good mixing using standard stirring equipment. In this batch process, when a combination of reagents are used, the oxidizing reagent can also be added at the beginning with the polymer and the hydrolytic reagent can be added at a later predetermined time in the process in order to achieve the desired results. The neutralization acid used to maintain the reaction in the desired pH range can be any acid, including hydrochloric acid, adipic acid, succinic acid, fumaric acid, malic acid etc.

Alternatively, the reaction with the oxidizing reagent can be conducted in a high-solids state without added water, or in the presence of low levels of water to give a wetted solid rather than an aqueous dispersion at the end of the reaction with the oxidizing agent. In this case, the wetted solid is then mixed with sufficient water to produce a fluid aqueous dispersion for removal of the water insoluble material.

In accordance with the invention, examples of methods useful in the process of removal of odorous components of the cationic galactomannan polymers of the invention include nitrogen sparging, distillation, adsorption, ion exchange, and membrane diafiltration or combinations thereof. Nitrogen sparging can be done at atmospheric pressure or with the aid of vacuum. Distillation in general could be employed or in this case, where the odiforous components are at such low levels (<100 ppm), extractive distillation, using water as the extractive solvent, would be more effective. Adsorbants, such as alumina, silica, or solid acids such as silica-aluminas, or acidic zeolites could be employed to remove basic odor-causing components. Alternatively, polystyrene-based Ion exchange resins could be similarly used to “scavenge” either acidic or basic compounds. Membranes could also be employed to remove low molecular weight impurities, regardless of the chemical characteristics. For instance, a nanofiltration membrane could be used to diafilter the low molecular weight cationic galactomannan polymer. Diafiltration is the process of washing low molecular weight compounds through the membrane with added water. The components that are washed through the membrane and those retained are dependant on the pore size of the membrane. In this diafiltration process, the level of removal of impurities increases with the volume of wash water that is employed. The membrane can be housed in a number of configurations, including hollow fiber, spiral wound, or plate and frame.

This invention is further directed to the use of the polygalactomannan composition of the present invention in functional systems such as personal care products, household care products, and pet care products. Other functional systems include industrial and institutional products, such as hand and body sanitizing products such as liquid soaps, can also be used in this invention. The above mentioned functional systems can optionally contain at least one other active personal care, household care, or pet care ingredient, respectively. In certain systems such as hair detangler liquids, gels, or sprays, the polygalactomannan itself can act as the active ingredient because of its affinity for the skin and hair. The functional systems of this invention can be oil-in-water or water-in-oil emulsions or solutions or slurries.

In accordance with the invention, examples of personal care products that may be incorporated into the polymer composition of the invention include cleansing and conditioning products such as two-in-one shampoos, three-in-one shampoos, shampoos, conditioners, shower gels, liquid soaps, bodywash formulas, styling products, shave gels/creams, body cleansers, and bar soaps.

In accordance with the present invention, the personal care active ingredient must provide some benefit to the user's body. Personal care products includes hair care, skin care, sun care, and oral care products. Examples of substances that may suitably be included in the personal care products according to the present invention are as follows:

1) Perfumes, which give rise to an olfactory response in the form of a fragrance and deodorant perfumes which in addition to providing a fragrance response can also reduce body malodor;

2) Skin coolants, such as menthol, menthyl acetate, menthyl pyrrolidone carboxylate N-ethyl-p-menthane-3-carboxamide and other derivatives of menthol, which give rise to a tactile response in the form of a cooling sensation on the skin;

3) Emollients, such as isopropylmyristate, silicone materials, mineral oils and vegetable oils which give rise to a tactile response in the form of an increase in skin lubricity;

4) Deodorants other than perfumes, whose function is to reduce the level of or eliminate micro flora at the skin surface, especially those responsible for the development of body malodor. Precursors of deodorants other than perfume can also be used;

5) Antiperspirant actives, whose function is to reduce or eliminate the appearance of perspiration at the skin surface;

6) Moisturizing agents, that keeps the skin moist by either adding moisture or preventing from evaporating from the skin;

7) Cleansing agents, that removes dirt and oil from the skin;

8) Sunscreen active ingredients, that protect the skin and hair from UV and other harmful light rays from the sun. In accordance with this invention a therapeutically effective amount will normally be from 0.01 to 10% by weight, preferable 0.1 to 5% by weight of the composition;

9) Hair treatment agents, that conditions the hair, cleans the hair, detangles hair, acts as styling agent, volumizing and gloss agents, anti-dandruff agent, hair growth promoters, hair dyes and pigments, hair perfumes, hair relaxer, hair bleaching agent, hair moisturizer, hair oil treatment agent, and antifrizzing agent;

10) Oral care agents, such as dentifrices and mouth washes, that clean, whiten, deodorize and protect the teeth and gum;

11) Denture adhesives that provide adhesion properties to dentures;

12) Shaving products, such as creams, gels and lotions and razor blade lubricating strips,

13) Tissue paper products, such as moisturizing or cleansing tissues;

14) Beauty aids, such as foundation powders, lipsticks, and eye care;

15) Textile products, such as moisturizing or cleansing wipes; and

16) Nail care products.

In accordance with the present invention, the household care and pet care active ingredient must provide some benefit to the user or pet. Examples of household and pet care products include dish detergents, fabric softeners, antistatic products, pet shampoo, deodorizing spray, and insect repellant products. Examples of active substances that may suitably be included according to the present invention are as follows:

1) Perfumes, which give rise to an olfactory response in the form of a fragrance and deodorant perfumes which in addition to providing a fragrance response can also reduce odor;

2) Insect repellent agent whose function is to keep insects from a particular area or attacking skin;

3) Bubble generating agent, such as surfactants which generates foam or lather;

4) Pet deodorizer such as pyrethrins which reduces pet odor;

5) Pet shampoo agents and actives, whose function is to remove dirt, foreign material and germs from the skin and hair surfaces;

6) Industrial grade bar, shower gel, and liquid soap actives that remove germs, dirt, grease and oil from skin, sanitizes skin, and conditions the skin;

7) All purpose cleaning agents, that remove dirt, oil, grease, germs from the surface in areas such as kitchens, bathroom, public facilities;

8) Disinfecting ingredients that kill or prevent growth of germs in a house or public facility;

9) Rug and Upholstery cleaning actives which lift and remove dirt and foreign particles from the surfaces and also deliver softening and perfumes;

10) Laundry softener actives which reduces static and makes fabric feel softer;

11) Laundry detergent ingredients which remove dirt, oil, grease, stains and kills germs and inhibit redeposition of substances;

12) Dishwashing detergents which remove stains, food, germs;

13) Toilet bowl cleaning agents which removes stains, kills germs, and deodorizes;

14) Laundry prespotter actives which helps in removing stains from clothes;

15) Fabric sizing agent which enhances appearance of the fabric;

17) Vehicle cleaning actives which removes dirt, grease, etc. from vehicles and equipment;

18) Lubricating agent which reduces friction between parts; and

19) Textile agents, such as dusting collection agents and cleaning agents.

The above list of personal care and household active ingredients are only examples and are not a complete lists of active ingredients that can be used. Other ingredients that are used in these types of products are well known in the industry. In addition to the above ingredients conventionally used, the composition according to the present invention can optionally also include ingredients such as a colorant, preservative, antioxidant, nutritional supplements, alpha or beta hydroxy acid, activity enhancer, emulsifiers, functional polymers, viscosifying agents (such as NaCl, NH4Cl, KCl, Na₂SO₄, fatty alcohols, fatty acid esters, fatty acid amides, fatty alcohol polyethyleneglycol ethers, sorbitol polyethyleneglycol ethers, cocamide monoethanolamide, cocamide diethanolamide, cocamidopropyl betaine, clays, silicas, cellulosic polymers, and xanthan), suspending agents (such as clays, silica, and xanthan), alcohols having 1-6 carbons, fats or fatty compounds, antimicrobial compound, zinc pyrithione, silicone material, hydrocarbon polymer, emollients, oils, surfactants, medicaments, flavors, fragrances, rejuvenating reagents, and mixtures thereof.

In accordance with the present invention, examples of functional polymers that can be used in blends with the cationic polygalactomannan or derivatives thereof of this invention include water-soluble polymers such as anionic, hydrophobically-modified, and amphoteric acrylic acid copolymers, vinylpyrrolidone homopolymers; cationic, hydrophobically-modified, and amphoteric vinylpyrrolidone copolymers; nonionic, cationic, anionic, and amphoteric cellulosic polymers such as hydroxyethylcellulose, hydroxypropylcellulose, carboxymethylcellulose, hydroxypropylmethylcellulose, cationic hydroxyethylcellulose, cationic carboxymethylhydroxyethylcellulose, and cationic hydroxypropylcellulose; acrylamide homopolymers and cationic, amphoteric, and hydrophobically-modified acrylamide copolymers, polyethylene glycol polymers and copolymers, hydrophobically-modified polyethers, hydrophobically-modified polyetheracetals, hydrophobically-modified polyols and polyetherurethanes and other polymers referred to as associative polymers, hydrophobically-modified cellulosic polymers, polyethyleneoxide-propylene oxide copolymers, and nonionic, anionic, hydrophobically-modified, amphoteric, and cationic polysaccharides such as xanthan, chitosan, carboxymethyl guar, alginates, hydroxypropyl guar, carboxymethyl guar hydroxypropyltrimethylammonium chloride, guar hydroxypropyltrimethylammonium chloride, hydroxypropyl guar hydroxypropyltrimethylammonium chloride.

In accordance with the invention, the silicone materials which can be used are, in particular, polyorganosiloxanes that are insoluble in the composition and can be in the form of polymers, oligomers, oils, waxes, resins, or gums.

The organopolysiloxanes are defined in greater detail in Walter Noll's “Chemistry and Technology of Silicones” (1968) Academic Press. They can be volatile or non volatile.

If volatile, the silicones are more particularly chosen from those having a boiling point of between 60° C. and 260° C., and even more particularly from:

(i) cyclic silicones containing from 3 to 7 and preferably from 4 to 5 silicon atoms. These are, for example, octamethylcyclotetrasiloxane sold in particular under the name “Volatile Silicone 7207” by Union Carbide or “Silbione 70045 V 2” by Rhone Poulenc, decamethyl cyclopentasiloxane sold under the name “Volatile Silicone 7158” by Union Carbide, and “Silbione 70045 V 5” by Rhone Poulenc, and mixtures thereof.

Mention may also be made of mixtures of cyclic silicones with organosilicone compounds, such as the mixture of octamethylcyclotetrasiloxane and tetratrimethylsilylpentaerythritol (50/50) and the mixture of octamethylcyclotetrasiloxane and oxy I,I′ bis(2,2,2′,2′,3,3′ hexatrimethylsilyloxy) neopentane;

(ii) linear volatile silicones having 2 to 9 silicon atoms and having a viscosity of less than or equal to 5×10−6 m2/s at 25° C. An example is decamethyltetrasiloxane sold in particular under the name “SH 200” by Toray Silicone Company. Silicones belonging to this category are also described in the article published in Cosmetics and Toiletries, Vol. 91, January 76, pp. 27 32, Todd & Byers “Volatile Silicone Fluids for Cosmetics”.

Non volatile silicones, and more particularly polyarylsiloxanes, polyalkylsiloxanes, polyalkylarylsiloxanes, silicone gums and resins, polyorganosiloxanes modified with organofunctional groups, and mixtures thereof, are preferably used.

In accordance with the invention, the silicone polymers and resins which can be used are, in particular, polydiorganosiloxanes having high number-average molecular weights of between 200,000 and 1,000,000, used alone or as a mixture in a solvent. This solvent can be chosen from volatile silicones, polydimethylsiloxane (PDMS) oils, polyphenylmethylsiloxane (PPMS) oils, isoparaffins, polyisobutylenes, methylene chloride, pentane, dodecane and tridecane, or mixtures thereof.

Examples of these silicone polymers and resins are as follows:
Polydimethylsiloxane,
polydimethylsiloxanes/methylvinylsiloxane gums,
polydimethylsiloxane/diphenylmethylsiloxane,
polydimethylsiloxane/phenylmethylsiloxane, and
polydimethylsiloxane/diphenylsiloxanemethylvinylsiloxane.

Products which can be used more particularly in accordance with the invention are mixtures such as:
(a) mixtures formed from a polydimethylsiloxane hydroxylated at the end of the
chain (referred to as dimethiconol according to the nomenclature in the CTFA dictionary) and from a cyclic polydimethylsiloxane (referred to as cyclomethicone according to the nomenclature in the CTFA dictionary), such as the product Q2 1401 sold by the Dow Corning Company;
(b) mixtures formed from a polydimethylsiloxane gum with a cyclic silicone,
such as the product SF 1214 Silicone Fluid from the company General Electric Company; this product is an SF 30 gum corresponding to a dimethicone, having a number average molecular weight of 500,000, dissolved in SF 1202 Silicone Fluid oil corresponding to decamethylcyclopentasiloxane; and
(c) mixtures formed of two PDMSs of different viscosities, and more particularly of a PDMS gum and a PDMS oil, such as the product SF 1236 from the General Electric Company. The product SF 1236 is a mixture of a gum SE 30 defined above, having a viscosity of 20 m2/s, and an oil SF 96, with a viscosity of 5×10−6 m2/s. This product preferably contains 15% SE 30 gum and 85% SF 96 oil.

These silicone materials in personal care and household products function as conditioning agents for hair, skin, and textile surfaces. Other types of conditioning agents include hydrocarbon oils, such as mineral oil and fatty acid ester of glycerol, and panthenol and its derivatives, such as panthenyl ethyl ether, panthenyl hydroxypropyl steardimonium chloride, and pantothenic acid.

For a more detailed understanding of the invention, reference can be made to the following examples which are intended as further illustration of the invention and are not to be construed in a limiting sense. All parts and percentages are by weight unless stated otherwise.

### EXAMPLES

In the following Examples the level of trimethylamine (TMA) was measured using two different methods. The limit of detection for Method 1 was ascertained to be 7 ppm. A second method, Method 2, was developed to measure lower levels of TMA in the low molecular weight cationic guar product. “PPM” means parts per million. “PPB” means parts per billion. All parts and percentages are by weight unless otherwise specified.

### Standard Methods and Procedures

Method 1 and/or Method 2 were used for analysis of the TMA level in all Examples shown in Tables 2, 3, and 4.

Method 1: Trimethylamine in Low Molecular Weight Cationic Guar by Headspace GC

The samples were prepared by weighing ˜0.5 g of the low molecular weight cationic guar solution into the headspace vial and then adding 5 ml of tris buffer at pH ˜8.5. The vials were equilibrated at ˜40° C. for 15 minutes prior to injection into the gas chromatography (GC) inlet and quantified using flame ionization detection (FID). Calibration was determined with external standards in equal volumes of the buffer. The detection limit was ˜7 ppm.

Method 2:Trimethylamine in Low Molecular Weight Cationic Guar by Headspace SPME/GC/FID

This method was used to determine trimethylamine (TMA) in cationic guar polymer solutions. The sample was adjusted to pH 8.5 with buffer in a headspace vial and TMA was extracted with a solid phase microextraction (SPME) fiber. TMA was desorbed from the fiber in a gas chromatography (GC) inlet and quantitated using flame ionization detection (FID). Calibration was determined with external standards. The result was reported as ug/g (ppm) to two significant figures.

Apparatus

(1) Gas Chromatograph, Agilent 6890A, equipped with a flame ionization detector (FID), a split/splitless injector, a 0.75 mm ID SPME liner (Restek cat. no. 21110), a Merlin Microseal GC inlet seal (Aldrich cat. no. 22581-U), a Gerstel MPS-2 SPME autosampler and an Agilent DB-Wax column, 30 m×0.53 mm×1.0 μm (VWR cat. no. 21512-352 ).

(2) SPME fiber assembly, 50/30 μm DVB/Carboxen/PDMS StableFlex for auto holder, gray (Aldrich cat. no. 57329-U).

Reagents

(1) Trimethylamine hydrochloride (TMA*HCL), reagent grade (98%), CAS# 593-81-7 (Aldrich cat. no. T72761).

(2) Water, reagent grade, CAS #7732-185 (VWR cat. no, 365-4).

(3) TRIS buffer (VWR cat. no. JTX171-5).

(4) o-Phosphoric acid, 85% (VWR cat. no. EM-552-3).

(5) TRIS buffer solution, pH 8.5 was prepared by adding 10.8 g of TRIS buffer to 500 mL reagent grade water and then titrated to pH 8.5 with concentrated phosphoric acid.

Calibration

(1) A stock calibration solution was prepared by weighing about 40 mg TMA*HCL in a 25-ml volumetric flask and recording the weight to the nearest 0.0001 g. The solution was brought to volume with reagent grade water and mix well. Use Equation (1) to calculate the concentration of TMA in the solution as μg/mL.

(2) A diluted stock calibration solution was prepared by pipetting 0.25 mL of the stock solution into a 25-mL volumetric flask and brought to volume with reagent grade water and mixed well.

(3) The following amounts of diluted stock solution were pipetted into 10-mL volumetric flasks and brought to volume with reagent grade water to prepare the working standards. Equation (2) was used to calculate the exact level of TMA in each standard.

| Standard # | Diluted Stock, mL |
|---|---|
| 1 | 0.5 |
| 2 | 1.0 |
| 3 | 1.5 |
| 4 | 2.0 |

(4) 1 mL of pH 8.5 TRIS buffer was pipetted into a 10-mL headspace vial and 1 mL of Standard #1 was pipetted into the vial. A headspace cap was crimped onto the vial immediately.

(5) Step (4) was repeated for the three remaining Standards.

(6) The four Standards were analyzed using the parameters listed for the MPS-2 auto sampler and the Agilent 6890A GC/FID.

(7) Excel software was used to calculate the slope and y-intercept for the four Standards with the concentrations as the x values and the areas as the y values. If ChemStation is available, it can be used instead of Excel to determine the calibration curve.

Procedure

(1) The volume of sample specified in the following Table 1 was pipetted into a 10-mL volumetric flask. The weigh was recorded to the nearest 0.0001 g. The sample was brought to volume with reagent grade water and mixed well. 1 mL of the sample solution was pipetted into a 10-mL headspace vial.

TABLE 1

| Expected ug/g | mL sample | Dilution Factor |
|---|---|---|
| 1 to 4 | 5 | 2 |
| 4 to 10 | 2 | 5 |
| 10 to 20 | 1 | 10 |
| 20 to 50 | 0.4 | 25 |
| 50 to 100 | 0.2 | 50 |

(2) 1 mL pH 8.5 TRIS buffer solution was pipetted into the vial and a headspace cap was crimped onto the vial immediately. The vial was gently swirled to mix.

(3) The sample was analyzed using the parameters listed for the MPS-2 auto sampler and the Agilent 6890A GC/FID.

(4) The TMA level was calculated using Equation (3) or using ChemStation according to the parameters listed for Agilent ChemStation. If the area of the sample is outside the calibration range, dilute the sample appropriately in water and rerun.

Gerstel MPS-2 Parameters

| Cycle SPME | |
|---|---|
| Syringe Fiber | |
| Pre-incubation Time 00:05:00 hr:min:sec | |
| Incubation Temp. | 35.0° C. |
| Agitator Speed | not used |
| Agitator On Time | Os |
| Agitator Off Time | Os |
| Vial Penetration | 22.0 mm |
| Extraction Time | 00:10:00 hr:min:sec |
| Desorb To | GC Injector |
| Injection Penetration | 54.0 mm |
| Desorb Time 00:05:00 hr:min:sec | |
| Fiber Bakeout | 00:00:00 hr:min:sec |

GC Runtime 00:20:00 hr:min:sec
Agilent 6890A Gas Chromatograph Operating Conditions
Injection Manual

Oven Initial Temperature 50° C.

Initial Time 0.00 min
Rate 20.00° C./min
Final Temperature 230° C.
Equilibration Time 0.50 min

Inlet (Split/Splitless) Mode Splitless

Temperature 270° C.
Pressure 4.00 psi
Gas Type Helium

Detector (FID) Temperature 250° C.

Makeup Gas Helium
Combined Flow 20.0 mL/min

Agilent ChemStation Parameters
Calibration Table Calculate External Standard Percent

| | |
|---|---|
| Based On | Peak Area |
| Curve Type | Linear |
| Origin | Ignore |
| Weight | Equal |

Standard Amount ng/μL (μg/mL)

Sequence Table Sample Amount g

| | |
|---|---|
| Multiplier | .01 |

Dilution Factor listed in Table 1 in the Procedure Section
Calculations

| | |
|---|---|
| $\frac{Wt Std \times P \times 0.617 \times 0.01 \times 1 \text{,} 000 \text{,} 000}{25} = TMA in stock , μ g / mL$ | Eq (1) |

where:

Wt Std = weight of TMA*HCl, g
P = purity of TMA*HCl, %
0.617 = ratio of TMA MW to TMA*HCl MW
0.01 = conversion factor for %
1,000,000 = conversion factor for μg to g
25 = dilution volume, mL

| | |
|---|---|
| $\frac{V ds \times C tma}{1000} = TMA in standard , μg / mL$ | Eq (2) |

where:

V ds = volume of diluted stock solution, mL
C tma = TMA in stock, μg/MI

| | |
|---|---|
| $\frac{( A spl - b ) \times DF}{m \times Wt Spl} = TMA , μg / g ( ppm )$ | Eq (3) |

where:

A spl = area of TMA peak for sample
b = y-intercept for area vs. TMA concentration
DF = dilution factor, from Table 1 in the Procedure section
m = slope for area vs. TMA concentration
Wt Spl = weight of sample solution, g

### Precursor Examples 1 and 2

The precursors for the present invention were prepared using the following procedure.

The following ingredients were added to a 1000 gallon glass lined reactor. The water, peroxide, and malic acid were added to the reactor with stirring. The cationic guar and sodium hydroxide were added to this mixture. The mixture was heated to a temperature of 85° C., until the viscosity of a sample of the reaction mixture reached the desired viscosity. At this time, the sodium metabisulfite was added. Adipic acid and Phenoxetol® product were added to the reaction product, and the reaction product was removed from the reactor.
Water: 390 gal water (initial)+350 gal water (fed w/slurry)
CatGuar: 800 lbs (88 to 91% TS)
Peroxide: 45.2 lbs (35%)
NaOH: 35 to 40 lbs (25%)
Malic Acid: 20.8 to 25 lbs added during slurry add'n
Sodium metabisulfite (SMBS): 17 to 23 lbs+optional addition
Adipic Acid: 4.5 lbs @ packout
Phenoxetol: 36.8 lbs

The reaction slurry was then subjected to a filtration step to remove water insoluble material using a diaphragm filter press.

As shown in Table 2, the level of trimethylamine (TMA) in the product of Example 1 was measured as 67 ppm using Method 1. The level of TMA in the product of Example 2 was measured as 64 ppm. An amount of 13.5 lbs of Nipasept® sodium was added to the product, with an aliquot of malic acid, to return the pH value to neutral.

The procedures that were used in the Examples to reduce or remove odorous components or low molecular weight components from the product are described in Examples 3-13.

### Examples 3-6

Adsorbents were used to remove odor components from the Precursors of Examples 1 and 2.

Examples 3-6 were prepared by adding ˜100 grams of low molecular weight cationic guar (LMWCG) to 4 ounce bottles and then for each treatment between 5-20 grams of adsorbent were added. Prior to treatment with charcoal, the pH of the LMWCG was adjusted to pH 8.5 using aqueous 5% NaOH. The adsorbent/LMWCG slurries were stirred with magnetic stirrer bars for approximately eight hours at 400 rpm. Then the bottles were placed in a rotating shaker for one hour at 30 cycles/minute. The contents of the bottles were allowed to settle and the adsorbent-treated LMWCG solutions were sampled by decanting the liquid from the solid adsorbent. The Dowex G-26 treated LMWCG solution was filtered through a porcelain Buchner funnel. The pH of a one ounce aliquot was adjusted to pH 8 using aqueous 5% NaOH. The pH of another aliquot was adjusted to pH 6 using 5% NaOH.

For the adsorbent treatments shown in Table 2, Examples 3-6, the trimethylamine level was reduced from 67 ppm trimethylamine in the untreated LMWCG solution to 59 ppm in the charcoal treated LMWCG and to <7 ppm in the Dowex G-26 (cationic exchange resin) and zeolite (Type H, ZSM-5) treated LMWCG.

### Example 7

A continuous column treatment of low molecular weight cationic guar with zeolite was conducted using a 1.3 inch internal diameter glass column that was packed with 262 g of Degussa ZSM-5 Type H zeolite ⅛th inch extrudate pellets. The 10 wt % aqueous solution of low molecular weight cationic guar product of Example 1 was pumped through the column at a rate of 20 g/min. A total of 1150 g of product was collected having a level of 2.5 ppm TMA as measured by Method 2.

### Example 8

A continuous column treatment of low molecular weight cationic guar solution was conducted using a 1.3 inch internal diameter glass column that was packed with 186 g (dry weight) of Rohm and Haas Amberlyst® 15 ion exchange resin beads (0.029 inch diameter). A 10 wt % aqueous solution of low molecular weight cationic guar, prepared according to the method in Example 1, was pumped through the column at a rate of 18 g/min. A total of 1000 g of product was collected, having a TMA level of 1.4 ppm, as measured by Method 2.

### Examples 9 and 10

Diafiltration of low molecular weight cationic guar product prepared according to the procedure in Example 1 through a New Logic VSEP (Vibrating SEParator) with a pore size of 200 Dalton at pressures between 200 and 300 psig, at temperatures between 25 and 45° C. The dilution water is acidic (malic acid) and also contains salt (NaCl). The product retentate collected contained a level of 0.64 ppm (Example 9) and 6.5 ppm trimethylamine (for Example 10), as measured by Method 2.

A reduced level of boron was measured in the product retentate relative to the untreated polymer in Example 1, as measured by diluting the polymer sample with 4% HNO₃ in DI (deionized) water. The diluted samples were analyzed by inductively coupled plasma—atomic-emission spectroscopy.

### Example 11

Removal of TMA from low molecular weight cationic guar solution was demonstrated using a Liqui-Cel® hollow-fiber membrane contactor using the following procedure. Liqui-Cel® and Celgard® are registered trademarks of Hoechst Celanese Corp.

The low molecular weight cationic guar solution was heated to 60° C. with stirring in a vessel, and the pH was adjusted to 9 with base. The cationic guar solution flowed into the shell-side of the contactor, while maintaining feed pressure at or below 20 psi. Nitrogen gas flowed into the tube-side of the contactor, maintaining the N₂ pressure at 20 psi or less. The cationic guar was allowed to continuously recirculate back into the heated vessel. The N₂ stream was passed through the contactor once and vented. This process was allowed to continue for up to 5 hours. The product was then cooled to 25° C. and the pH adjusted to 6 with malic acid.

### Example 12

Nitrogen sparging of aqueous solutions of low molecular weight cationic guar reduced the level of TMA in the product as shown in Table 2, Example 12. The aqueous solution of cationic guar was heated to 60° C. in a stirred flask, the pH was adjusted to 8.5, and nitrogen sparging was performed for 2 hours at this temperature. The level of TMA in the product was reduced from 67 ppm to 16 ppm using this process.

### Example 13

It has been found that the level of amine odor and the level of TMA in aqueous solutions of low molecular weight cationic galactomannan polymers are significantly reduced when combined enzyme-peroxide processing is used to reduce the molecular weight of the cationic galactomannan polymer, instead of the peroxide oxidation process. The GC headspace analyses shown in Table 2, Example 13 also demonstrate the reduction in TMA levels in aqueous solutions of cationic galactomannan polymers that have been prepared using enzyme-peroxide combined processing (Example 13) compared with the peroxide process (Example 1).

TABLE 2
Effect of Treatment on Trimethylamine (TMA) Concentration in Low Molecular
Weight Cationic Guar (Polygalactomannan) Solutions (10 weight percent total solids).

| | | Adsorbent | | | | |
|---|---|---|---|---|---|---|
| | Treatment | Concentration | Boron/ | Wt % | TMA/ppm | |
| Example | Description | (weight percent) | wt % | Protein | Method 1 | TMA/ppm Method 2 |
| Example 1 | None | — | 36 | 0.38 | 67 | |
| | Comparative | | | | | |
| | Control | | | | | |
| Example 2 | None (control) | — | | | | 64 |
| Example 3 | Dowex G26, pH 8, | 16.5 | | 0.37 | Not | |
| | Ion exchange resin | | | | detected | |
| | | | | | <7 ppm* | |
| Example 4 | Dowex G26, pH 6, | 16.5 | | | Not | |
| | Ion exchange resin | | | | detected | |
| | | | | | <7 ppm* | |
| Example 5 | Zeolite | 9.1 | | 0.199 | Not | |
| | Type H-ZSM-5 | | | | detected | |
| | | | | | <7 ppm* | |
| Example 6 | Charcoal, pH 8.5 | 4.5 | | | 59 | |
| Example 7 | Degussa ZSM-5 | Column | | | | 2.5 |
| | Type-H Zeolite Type | | | | | |
| | 1/8 in. extrudate | | | | | |
| | pellets | | | | | |
| Example 8 | Amberlyst ® 15 | Column | | | | 1.4 |
| | 0.029 in. diameter | | | | | |
| | Rohm and Haas | | | | | |
| Example 9 | Diafiltration | — | <5 | | <7 ppm* | 0.64 |
| | pH 5.8 | | | | | |
| Example 10 | Diafiltration | — | | 0.44 | | 6.5 |
| | pH 5.8 | | | | | |
| Example 11 | Hollow Fiber | — | | | | |
| | Membrane | | | | | |
| Example 12 | Nitrogen Sparging, | — | | | 16 ppm | |
| | pH 9, 2 hrs, 60° C. | | | | | |
| Example 13 | Mannanase | — | | 0.81 | Not | |
| | enzyme-peroxide | | | | detected | |
| | degradation | | | | <7 ppm* | |

7 ppm was the detection limit for analytical Method 1

The analyses shown in Examples 3-13 compared with Example 1 and 2 in Table 2 demonstrate the reduction in the level of trimethylamine (TMA) in aqueous solutions of a cationic guar subjected to these post-treatments.

In accordance with the invention, in a preferred process, the molecular weight reduction step is conducted in aqueous medium to produce a dispersion, and water insoluble solids are removed from the dispersion, and one of the processes shown in the Examples 3 through 12 in Table 2 is applied, to produce a clarified, low odor solution of the galactomannan polymer composition of the invention. Optionally, water soluble color bodies are removed to make a colorless, clarified, low odor, aqueous solution of the cationic galactomannan polymer or derivatized cationic galactomannan polymer. Optionally, the resultant cationic galactomannan polymer or derivatized cationic galactomannan polymer can also be recovered in dry form from solution.

### Examples 14-16

The reduced perception of odor in personal care compositions containing the low odor cationic polygalactomannan samples of the invention are demonstrated in the following Examples and the results are reported in Tables 3 and 4. The procedure for preparing the shampoo formulations used in Table 3 is described in the following procedure.

Procedure: Conditioning Shampoo

Shampoo formulations were prepared, containing low molecular weight cationic guar of the invention, an untreated cationic guar, or a control shampoo in which water was substituted for the cationic guar solution. The aqueous solutions of cationic guar were adjusted for their total solids content and the water charge was adjusted accordingly. Two samples of each formulation were made, one sample adjusted to pH 5.5-6.0 and one sample adjusted to pH 8.0-8.5.

Shampoo Formulation:

| Examples | | |
|---|---|---|
| 14 | 15 | 16 |

Part A

| | | | |
|---|---|---|---|
| Deionized water | 65.56 | 65.56 | 65.56 |
| Hydroxyethylcellulose (HEC) | 1.04 | 1.04 | 1.04 |
| 1.0% NaOH solution | q.s. | q.s. | q.s. |

Part B

| | | | |
|---|---|---|---|
| Sodium lauryl sulfate (SLS) | 17.00 | 17.00 | 17.00 |
| Sodium laureth sulfate (SLES) | 13.00 | 13.00 | 13.00 |
| Cocamindopropylbetaine (CAPB) | 2.50 | 2.50 | 2.50 |
| DMDM hydantoin | 0.50 | 0.50 | 0.50 |

Part C

| | | | |
|---|---|---|---|
| Deionized water | 0.40 | 0.00 | 0.00 |
| Cationic guar¹ | 0.00 | 0.40 | 0.00 |
| Cationic guar² | 0.00 | 0.00 | 0.40 |
| | 100.00 | 100.00 | 100.00 |

¹Polymer of Example 2 AquaCat CG-518, marketed by Hercules Incorporated
²Polymer of Example 10

Part A—Deionized water was charged to the mixing vessel, HEC was added while mixing, and the mixture was stirred for 10 minutes to disperse. The pH of mixture was adjusted to 8.0-8.5 with 1.0% NaOH solution. The mixtured was then stirred for 30 minutes, then the pH was re-adjusted to 8.0-8.5, and the mixture was continued stirring for an additional 30 minutes.
Part B—Order of addition to Part A while mixing: SLES, CAPB, Methyl gluceth-20, DMDM hydantoin. Mixed 90 minutes.
Part C—Added AquaCat or water to Parts A and B while mixing, mixed 15 minutes.
Shampoo at pH 5.5-6.0 X33768-76 A-1, -76 B-1, -76 C-1: Adjusted shampoo to pH 5.5-6.0 with 5.0% Citric acid solution, mixed 15 minutes.

Shampoo at pH 8.0-8.5 X33768-76 A-2. -76 B-2. -76 C-2: Adjusted shampoo to pH 8.0-8.5 with 1.0% NaOH solution, mixed 15 minutes.

| Material | Trademark | Supplier | |
|---|---|---|---|
| Hydroxyethylcellulose | Natrosol ® 250HHR-CS | Hercules Inc. | 96.45% active |
| Sodium laureth sulfate | Rhodapex ® ES-STD | Rhodia | 27% active (3-EO) |
| Cocamidopropylbetaine | Amphosol ® CA | Stepan Co. | 30% active |
| Methyl gluceth-20 | Glucam ® E-20 | Amerchol Corp. | 100% |
| DMDM Hydantoin | Glydant ® | Lonza Group | 100% active |
| Cationic guar solution | AquaCat ® CG-518 | Hercues Inc. | 10.11% active |
| cationic guar solution | AquaCat ® CG-518 | | |

TABLE 3
Odor Panel Assessment of Shampoo Formulations at Acidic and Alkaline pH Values

| | | | | | TMA/ | Odor Ranking of Shampoos at pH 5.5 and 8.5 by Panelists 1, 2, and 3 | | | | | |
|---|---|---|---|---|---|---|---|---|---|---|---|
| | | | | | ppm | pH | pH | pH | pH | pH | pH |
| | | | | Polymer/ | In | 5.5 | 8.5 | 5.5 | 8.5 | 5.5 | 8.5 |
| Polymer | Example | DS | Mw | wt % | Polymer | Panelist 1 | Panelist 1 | Panelist 2 | Panelist 2 | Panelist 3 | Panelist 3 |
| Control- | 14 | - - - | - - - | 0 | 0 | None | None | None | None | Slight-not | Moderate soapy- |
| water | | — | | | | | | | | offensive | not offensive |
| Example | 15 | 0.2 | 39,900 | 0.4% | 6.5 | None | None | None | None | Moderate | Moderate soapy- |
| 10 | | | | | | | | | | not | not offensive |
| | | | | | | | | | | offensive | |
| Example 2 | 16 | 0.2 | 41,800 | 0.4% | 64 | None | Slight | Slight- | Stronger | Moderate | Considerable fishy, |
| | | | | | | | fishy | moderate | amine | not | offensive |
| | | | | | | | | amine | | offensive | |

Odor panelists were asked to open each sample and assess the headspace for odor for 15 seconds. As shown by the results in Table 3, as expected, in shampoos formulated at a pH of 5.5-6.0, only one of the panel members detected a slight amine odor in the shampoo formulated with the untreated polymer of Example 2, which had a measured TMA level of 64 ppm.

In shampoos formulated at a pH of 8.0-8.5, all panel members detected amine, fishy, or offensive odor in shampoos formulated with the untreated polymer of Example 2, which had a measured TMA level of 64 ppm. None of the panel members detected amine, fishy, or offensive odor in the shampoo formulated with the polymer of the invention, Example 10, which had a measured TMA level of 6.5 ppm.

### Examples 17-19

Bodywash formulations were prepared, containing low molecular weight cationic guar of the invention, an untreated cationic guar, or a control in which water was substituted for the cationic guar solution. The aqueous solutions of cationic guar were adjusted for their total solids content and the water charge was adjusted accordingly. Two samples of each formulation were made, one sample adjusted to pH 5.5-6.0 and one sample adjusted to pH 8.0-8.5. The procedure and bodywash formulation are described below.

Procedure: Body Wash at pH 5.5-6.0

A large batch of the body wash formulation was prepared in which 5.0% of the water charge was held out in order to allow for the addition of the various cationic guar polymer solutions. Body washes containing 0.40% active cationic guar were prepared by adding 5.0 ppw (parts per weight) of the aqueous cationic guar solution to 95 ppw of the bodywash stock solution. Three body wash formulations were prepared, containing either untreated low Mw cationic guar of Example 2, the treated cationic guar polymer of the invention, Example 10, or a control body wash in which water was substituted for the cationic guar solution. The polymer charges were adjusted for their total solids content and the water charge was adjusted accordingly. Two samples of each formulation were made, one sample adjusted to pH 5.5-6.0 and one sample adjusted to pH 8.0-8.5.

Body Wash Stock Solution:

Part A

| | |
|---|---|
| Deionized water | 47.96 |
| Hydroxyethylcellulose (HEC) | 1.04 |
| 1.0% NaOH solution | q.s. |

Part B

| | |
|---|---|
| Sodium laureth sulfate (SLES) | 42.00 |
| Cocamindopropylbetaine (CAPB) | 3.00 |
| Methyl gluceth-20 | 0.50 |
| DMDM hydantoin | 0.50 |
| | 90.00 |

Part A—Deionized water was charged to the mixing vessel, HEC was added while mixing, and the mixture was stirred 10 minutes to disperse. The pH of mixture was adjusted to 8.0-8.5 with 1.0% NaOH solution. The mixture was stirred for an additional 30 minutes; then the pH of the mixture was re-adjusted to 8.0-8.5; and the mixture was continued mixing for an additional 30 minutes.

Part B—Order of additions to Part A while mixing: SLES, CAPB, Methyl gluceth-20, DMDM hydantoin. Mixed 90 minutes.

Body Wash at pH 5.5-6.0 Examples 17, 18, 19

| | |
|---|---|
| Stock solution | 95.00 |
| Cationic guar solution | 5.00 |
| Deionized water | q.s. |
| 5.0% Citric acid solution | q.s. |
| | 100.00 |

The cationic guar solutions and/or water were added while mixing the bodywash stock solution and were mixed for 15 minutes. The samples were adjusted to pH 5.5-6.0 with 5.0% citric acid and mixed another 15 minutes.

Body Wash at pH 8.0-8.5: Examples 17, 18, 19

| | |
|---|---|
| Stock solution | 95.00 |
| Cationic guar solution | 5.00 |
| Deionized water | q.s. |
| 1.0% NaOH solution | q.s. |
| | 100.00 |

The cationic guar solutions and/or water were added while mixing to the bodywash stock solution and mixed for 15 minutes. The samples were adjusted to pH 8.0-8.5 with 1.0% NaOH solution and mixed another 15 minutes.

TABLE 4
Odor Panel Assessment of Bodywash Formulations at Acidic and Alkaline pH Values

| | | | | | TMA/ | Odor Ranking of Bodywash at pH 5.5 and 8.5 by Panelists 1, 2, and 3 | | | | | |
|---|---|---|---|---|---|---|---|---|---|---|---|
| | | | | | ppm | pH | pH | pH | pH | pH | pH |
| | | | | Polymer/ | In | 5.5 | 8.5 | 5.5 | 8.5 | 5.5 | 8.5 |
| Polymer | Example | DS | Mw | wt % | Polymer | Panelist 1 | Panelist 1 | Panelist 2 | Panelist 2 | Panelist 3 | Panelist 3 |
| Control- | 17 | - - - | - - - | 0 | 0 | None | None | None | None | Slight-not | Slight soapy- |
| water | | — | | | | | | | | offensive | not offensive |
| Example | 18 | 0.2 | 39,000 | 0.4% | 6.5 | None | None | None | Trace | Slight | Moderate soapy- |
| 10 | | | | | | | | | Amine | soapy | not offensive |
| | | | | | | | | | | not | |
| | | | | | | | | | | offensive | |
| Example 2 | 19 | 0.2 | 41,800 | 0.4% | 64 | None | Slight | None | Slight | Moderate | Considerable amine, |
| | | | | | | | fishy | | amine | soapy not | offensive |
| | | | | | | | | | | offensive | |

Odor panelists were asked to open each sample and assess the headspace for odor for 15 seconds. As shown by the results in Table 4, as expected, in bodywash formulated at a pH of 5.5-6.0, none of the panel members detected amine, fishy, or offensive odor, even in the bodywash Example 19 formulated with the untreated polymer of Example 2, which had a measured TMA level of 64 ppm.

In bodywash formulated at a pH of 8.0-8.5, all panel members detected amine, fish, or offensive odor in bodywash Example 19, formulated with the untreated polymer of Example 2, which had a measured TMA level of 64 ppm. Only one of the panel members detected amine, fishy, or offensive odor in the bodywash Example 18 formulated with the polymer of the invention, Example 10, which had a measured TMA level of 6.5 ppm.

The combined results in Tables 3 and 4 demonstrate that personal care formulations at alkaline pH values containing the treated polymer of the invention. Example 10, have significantly reduced odor than personal care products containing untreated polymer, Example 2.

The samples of low molecular weight cationic guar polymer of the invention having reduced odor also show reduced odor in shampoo formulations, bodywash formulations, and other personal care products formulated at alkaline pH values. This same reduced odor performance for these reduced odor products are expected in household products, and pet care products that are formulated at alkaline pH values.

While the invention has been described with respect to specific embodiments, it should be understood that the invention should not be limited thereto and that many variations and modifications are possible without departing from the spirit and scope of the invention.

What is claimed:

1. A reduced odor composition comprising at least one cationic polygalactomannan or a derivative of cationic polygalactomannan having a weight average molecular weight (Mw) having a lower limit of 5,000 and an upper limit of 200,000 and having a light transmittance in a 10% aqueous solution of greater than 80% at a light wavelength of 600 nm and a protein content of less than 1.0% by weight of polysaccharide, and a trimethylamine content of less than 25 ppm in a 10% aqueous solution of the polymer.

2. The composition of claim 1, wherein the composition has an aldehyde functionality content of at least 0.01 meq/gram.

3. The composition of claim 1, wherein the composition has a boron content of less than 100 ppm per gram of polygalactomannan.

4. The composition of claim 1, wherein the composition has a cationic degree of substitution (DS) lower limit of about 0.01 and an upper limit of about 3.0.

5. The composition of claim 4, wherein the cationic degree of substitution (DS) has a lower limit amount of 0.1 cationic DS.

6. The composition of claim 4, wherein the cationic degree of substitution (DS) has a lower limit amount of 0.2 cationic DS.

7. The composition of claim 4, wherein the cationic degree of substitution (DS) has an upper limit of about 2.0.

8. The composition of claim 4, wherein the cationic degree of substitution (DS) has an upper limit of about 1.0.

9. The composition of claim 1, wherein the derivative moiety on the cationic derivatized polygalactomannan is selected from the group consisting of alkyl, hydroxyalkyl, alkylhydroxyalkyl, and carboxymethyl wherein the alkyl has a carbon chain containing from 1 to 22 carbons and the hydroxyalkyl is selected from the group consisting of hydroxyethyl, hydroxypropyl, and hydroxybutyl.

10. The composition of claim 1, wherein the polygalactomannan is selected from the group consisting of guar, locust bean, honey locus, and flame tree.

11. The composition of claim 1, wherein the cationic moiety is selected from quaternary ammonium compounds.

12. The composition of claim 11, wherein the quaternary ammonium compound is selected from the group consisting of 3-chloro-2-hydroxypropyltrimethylammonium chloride, 2,3-epoxy-propyltrimethylammonium chloride, 3-chloro-2-hydroxypropyltrimethylammonium bromide, 2,3-epoxy-propyltrimethylammonium bromide; glycidyltrimethylammonium chloride, glycidyltriethylammonium chloride, gylcidyltripropylammonium chloride, glycidylethydimethylammonium chloride, glycidyldiethylmethylammonium chloride, and their corresponding bromides and iodides; 3-chloro-2-hydroxypropyltrimethylammonium chloride, 3-chloro-2-hydroxypropyltriethylammonium chloride, 3-chloro-2-hydroxypropyltripropylammonium chloride, 3-chloro-2-hydroxypropylethyldimethylammonium chloride, and their corresponding bromides and iodides; and halides of imidazoline ring containing compounds.

13. The composition of claim 1, wherein the light transmittance is greater than 90%.

14. The composition of claim 1, wherein the light transmittance is greater than 95%.

15. The composition of the claim 1, wherein the protein content in the composition is less than about 0.5% by weight of polysaccharide.

16. The composition of claim 1, wherein the Mw has a lower limit of 20,000.

17. The composition of claim 1, wherein the Mw has a lower limit of 35,000.

18. The composition of claim 1, wherein the Mw has a lower limit of 50,000.

19. The composition of claim 1, wherein the Mw has an upper limit of 100,000.

20. The composition of claim 1, wherein the Mw has an upper limit of 70,000.

21. The composition of claim 1, further comprising a member selected from the group consisting of colorant, preservative, antioxidant, alpha or beta hydroxy acid, activity enhancer, emulsifier, functional polymer, viscosifying agent, alcohol, fat or fatty compound, antimicrobial compound, zinc pyrithione, silicone material, hydrocarbon polymer, emollients, oil, surfactants, suspending agents, and mixtures thereof.

22. The composition of claim 21, wherein the functional polymer is selected from the group consisting of anionic, hydrophobically-modified, and amphoteric acrylic acid copolymers, vinylpyrrolidone homopolymers and copolymers, cationic vinylpyrrolidone copolymers, nonionic, cationic, anionic, and amphoteric cellulosic polymers, acrylamide homopolymers, cationic, anionic, amphoteric, and hydrophobically-modified acrylamide copolymer, polyethylene glycol polymer and copolymer, hydrophobically-modified polyether, hydrophobically-modified polyetheracetal, hydrophobically-modified polyetherurethane, an associative polymer, hydrophobically-modified cellulosic polymer, polyethyleneoxide-propylene oxide copolymer, and a nonionic, anionic, hydrophobically-modified, amphoteric, and cationic polysaccharides, chitosan, and mixtures thereof.

23. The composition of claim 22, wherein the nonionic, cationic, anionic, and amphoteric cellulosic polymers are selected from the group consisting of hydroxyethylcellulose, hydroxypropylcellulose, hydroxypropylmethylcellulose, carboxymethylcellulose, hydrophobically-modified carboxymethylcellulose, cationic hydroxyethylcellulose, cationic hydrophobically-modified hydroxyethyl cellulose, hydrophobically modified hydroxyethylcellulose, hydrophobically-modified hydroxypropylcellulose, cationic hydrophobically-modified hydroxypropyl cellulose, cationic carboxymethylhydroxyethylcellulose, and cationic hydroxypropylcellulose.

24. The composition of claim 22, wherein the nonionic, anionic, hydrophobically modified, amphoteric, and cationic polysaccharides are selected from the group consisting of carboxymethyl guar, alginates, hydroxypropyl guar, hydrophobically-modified guar, carboxymethyl guar hydroxypropyltrimethylammonium chloride, guar hydroxypropyltrimethylammonium chloride, and hydroxypropyl guar hydroxypropyltrimethylammonium chloride.

25. The composition of claim 21, wherein the viscosifying agent is selected from the group consisting of NaCl, NH₄Cl, KCl, Na₂SO₄, fatty alcohols, fatty acid esters, fatty acid amides, fatty alcohol polyethyleneglycol ethers, sorbitol polyethyleneglycol ethers, cocamidopropyl betaine, clays, silicas, cellulosic polymers, xanthan, and mixtures thereof.

26. The composition of claim 21, wherein the silicone material is selected from the group consisting of cyclosiloxane, linear siloxane, siloxane structure with polyol, amino, or other functional groups in the siloxane structure, and mixtures thereof.

27. The composition of claim 26, wherein the other functional groups are selected from the group consisting of polyethyleneoxy and/or polypropyleneoxy groups optionally containing C₆-C₂₄ alkyl groups, substituted or unsubstituted amine groups, thiol groups, alkoxylated groups, hydroxyl groups, acyloxyalkyl groups.

28. The composition of claim 21, wherein the silicone material is selected from the group consisting of polyalkylsiloxanes, polyarylsiloxanes, polyalkylarylsiloxanes, and mixtures thereof.

29. The composition of claim 28, wherein the polyalkylsiloxanes are selected from the group consisting of polydimethylsiloxane, polydimethylsiloxane hydroxylated at the end of the chain, and mixtures thereof.

30. The composition of claim 21, wherein the surfactant is anionic, amphoteric, or nonionic.

31. A process comprising (a) reacting at least one cationic polygalactomannan or cationic derivatized polygalactomannan with at least one reagent that reduces the Mw to less than 200,000 that includes water soluble color bodies and water insoluble material, (b) removing water insoluble material, and c) removing odorous components, including trimethylamine(TMA) and other amines and low molecular weight components in an aqueous phase to produce the composition of claim 1.

32. The process of claim 31, wherein the cationic polyglactomannan or cationic derivatized polygalactomannan is treated with the reagent in aqueous medium to produce an aqueous dispersion composed of an aqueous phase and a solid phase of the treated polygalactomannan, the water insoluble solid materials are removed from the dispersion eliminating the solid phase, and odorous components, including trimethylamine (TMA) and other amines and low molecular weight components are removed from the aqueous phase to produce a clarified solution of the composition of claim 1.

33. The process of claim 32, wherein the reagent is an oxidizing reagent selected from the group consisting of peroxides, persulfates, permanganates, perchlorates, hypochlorite, oxygen, and biochemical oxidants.

34. The process of claim 33, wherein the peroxide is hydrogen peroxide.

35. The process of claim 33, wherein the biochemical oxidizing reagent is an oxygenase.

36. The process of claim 35, wherein the oxygenase is galactose oxidase.

37. The process of claim 32, wherein the reagent further comprises a hydrolytic reagent.

38. The process of claim 37, wherein said hydrolytic reagent is selected from the group considering of hydrolytic enzymes.

39. The process of claim 38, wherein said hydrolytic enzyme is selected from the group consisting of hemicellulases.

40. The process of claim 39, wherein the hemicellulase is mannanase.

41. The process of claim 37, wherein said hydrolytic reagent is an organic or mineral acid.

42. The process of claim 31, further comprising removing the water soluble color bodies.

43. The process of claim 31, further comprising removing the water soluble color bodies to produce a colorless, clarified aqueous solution.

44. The process of claim 42, wherein the water soluble color bodies are removed by addition of sodium metabisulfite, sodium bisulfite, sodium hypochlorite, or sodium chlorite.

45. The process of claim 42, wherein the water soluble color bodies are removed by addition of activated carbon, followed by a separation step.

46. The process of claim 42, wherein the water soluble color bodies are removed by addition of molecular sieves, followed by a separation step.

47. The process of claim 32, further comprising recovering the derivatized polygalactomannan in dry form from the aqueous solution.

48. The process of claim 31, wherein the cationic polygalactomannan or cationic derivatized polygalactomannan is in the form of powder, flour, or splits.

49. The process of claim 31, wherein the removal step of odorous components, includes processes selected from the group consisting of nitrogen sparging, distillation, adsorption, ion exchange, and membrane diafiltration.

50. The process of claim 49, wherein the membrane diafiltration uses nanofiltration membranes selected from the group consisting of hollow fiber, spiral wound, and plate and frame.

51. The process of claim 49, wherein the adsorption process includes adsorbents selected from the group consisting of alumina, silica, silica-aluminas, and acidic zeolites.

52. The process of claim 49, wherein the ion exchange process uses polystyrene-based ion exchange resins.

53. The process of claim 49, wherein the nitrogen sparging is performed at atmospheric pressure or with the aid of vacuum.

54. The process of claim 49, wherein the distillation process includes general distillation or extractive distillation, using water as the extractive solvent.

55. A composition comprising a functional system selected from the group consisting of personal care product, household product, and pet care product containing the composition of claim 1.

56. The composition of claim 55, wherein the composition of claim 1 is incorporated into the functional system in an amount of less than 10 wt %.

57. The composition of claim 55, wherein the composition of claim 1 is incorporated into the functional system in an amount of less than 5 wt %.

58. The composition of claim 55, wherein the composition of claim 1 is incorporated into the functional system in an amount of less than 1 wt %.

59. The composition of claim 55, wherein the functional system has an acidic pH.

60. The composition of claim 55, wherein the functional system has an neutral pH.

61. The composition of claim 55, wherein the functional system has an alkaline pH.

62. The composition of claim 55, wherein the functional system is a household care or pet care product.

63. The household care or pet care product composition of claim 62, further comprising at least one active household care or pet care ingredient.

64. The household care or pet care product composition of claim 63, wherein the active household ingredient is selected from the group consisting of insect repellent agent, pet deodorizer agent, pet shampoo active, industrial grade bar and liquid soap active, dishwashing soap active, all purpose cleaner, disinfecting agent, rug and upholstery cleaning active, laundry softener active, laundry detergent active, toilet bowl cleaning agent, fabric sizing agent, dust collection agent, antiredeposition agent, textile cleaning agent, and lubricating agent.

65. The household care or pet care product composition of claim 62, wherein the composition also includes at least one additional ingredient selected from the group consisting of colorant, preservative, antioxidant, bleaching agent, activity enhancer, emulsifier, functional polymer, viscosifying agent, alcohol, fat or fatty compound, oil, surfactant, fragrance, suspending agent, silicone material, and mixtures thereof.

66. The composition of claim 55, wherein the functional system is a personal care product comprising the composition of claim 1.

67. The personal care composition of claim 66, further comprising at least one active personal care ingredient.

68. The personal care composition of claim 67, wherein the active personal care ingredient is selected from the group consisting of perfumes, skin coolants, emollients, deodorants, antiperspirants actives, moisturizing agents, cleansing agents, sunscreen actives, hair treatment agents, oral care agents, denture adhesive agents, shaving actives, beauty aids, and nail care active.

69. The personal care composition of claim 66, wherein the composition is a product selected from the group consisting of hair care, skin care, sun care, nail care, and oral care.

70. The personal care composition of claim 69, wherein the product is a hair care product comprising a conditioning agent selected from the group consisting of silicone materials, hydrocarbon oils, panthenol and derivatives thereof, pantothenic acid and derivatives thereof, and mixtures thereof.

71. The personal care composition of claim 69, wherein the product is a skin care product comprising a conditioning agent selected from the group of consisting of silicone materials, hydrocarbon oils, panthenol and derivatives thereof, pantothenic acid and derivatives thereof, and mixtures thereof.

72. The personal care composition of claim 69, wherein the product is a hair care product or skin care product comprising up to 5% by weight the composition of claim 1 and has a light transmittance value greater than 95%.

73. The personal care composition of claim 66, wherein the composition also includes at least one additional ingredient selected from the group consisting of colorant, preservative, antioxidant, alpha or beta hydroxy acid, activity enhancer, emulsifier, functional polymer, viscosifying agent, alcohol, fat or fatty compound, antimicrobial compound, zinc pyrithione, silicone material, hydrocarbon polymer, emollient, oil, surfactant, flavor, fragrance, medicaments, rejunvenating agents, suspending agents, and mixture thereof.

74. The composition of claim 55, wherein the functional system is an oil-in-water or water-in-oil emulsion.

75. The composition of claim 1, further comprising water in an amount of 50 - 95% by weight of the total composition.

76. The composition of claim 75, further comprising at least one additional ingredient selected from the group consisting of stabilizing biocides, fragrances, flavors, colorants, and mixtures thereof.
